(19) United States
(12) Patent Application Publication
(10) Pub. No.: US 2026/0207911 A1
(43) Pub. Date: Jul. 23, 2026

(54) MEDICAL INSTRUMENT AND MEDICAL DEVICE

(71) Applicant: FURUKAWA ELECTRIC CO., LTD., Tokyo (JP)

(72) Inventors: Yoshiki NOMURA, Tokyo (JP); Junichi HASEGAWA, Tokyo (JP)

(21) Appl. No.: 19/139,880

(22) PCT Filed: Dec. 22, 2023
(86) PCT No.: PCT/JP2023/046129

§ 371 Date: Jun. 17, 2025

(30) Foreign Application Priority Data
Dec. 23, 2022 (JP) 2022-206944

(51) Int. Cl. A61M 39/02

(52) U.S. Cl. CPC A61M 39/0208; A61M 2039/0238; A61M 2205/587

(57) ABSTRACT

Provided are a medical instrument and a medical device that ensure an installation surface for a light-emitting part but reduce the size of a body part in the radial direction. A medical instrument 10 that is to be embedded in a living body comprises a body part 11 that has a recess 11a that opens upward and extends in the thickness direction and can hold liquid in the recess 11a, a lid part 12 that comprises a soft member that can be pierced by an injection needle and closes the opening of the recess 11a, a power reception coil 13 that receives power transmitted from a power transmission coil 22a, and a circuit board 14 that is arranged on the outer circumferential side of the recess 11a of the body part 11 and has provided to one surface thereof a light-emitting part 14a that emits light using power received by the power reception coil 13. The circuit board 14 extends along the outer circumferential side of the recess 11a such that the surface to which the light-emitting part 14a is provided is directed toward the outside of the body part 11 in the radial direction.

### TECHNICAL FIELD

The present invention relates to a medical device and a medical instrument that is implanted in a living body.

### BACKGROUND ART

A conventionally known medical instrument to be implanted in a living body is provided with a main body that has a recessed section extending in the thickness direction and having an upper surface with an opening and that is capable of storing a liquid in the recessed section, a lid part that is formed from a soft member through which an injection needle can be inserted and that closes the opening of the recessed section of the main body, a power reception coil that receives power sent from a power transmission coil, and a circuit board that is disposed in the main body at a position on the outer circumference side of the recessed section and has one surface on which a light-emitting unit for emitting light by means of the power received by the power reception coil is provided (see, for example, Patent Document 1).

Bringing an outside-of-living-body unit having the power transmission coil close to the medical instrument implanted in a living body causes power to be transmitted from the power transmission coil to the power reception coil and thus causes the light-emitting unit to emit light, so that the position of the lid part can be identified outside the living body by means of the light emitted from the light-emitting unit.

### CITATION LIST

### Patent Document

Patent Document 1: PCT International Publication No. WO2022/102393

### DISCLOSURE OF THE INVENTION

### Problems to be Solved by the Invention

A medical instrument to be implanted in a living body is configured such that: a circuit board installed in a main body extends in the radial direction of the main body; and a light-emitting unit is installed on the upper surface of the circuit board extending in the radial direction of the main body. Thus, the main body of the medical instrument is formed so as to have such a radial size that the main body can have attached thereto a circuit board on which the light-emitting unit can be installed. Meanwhile, the main body of the medical instrument to be implanted in a living body needs to have a reduced radial size in order to reduce the invasiveness in the living body.

An object of the present invention is to provide a medical instrument and a medical device that can ensure an installation surface for a light-emitting unit and allow a main body to have a reduced radial size.

### Means for Solving the Problems

A medical instrument according to the present invention is implanted in a living body and includes a main body that has a recessed section extending in the thickness direction and having an upper surface with an opening and that is capable of storing a liquid in the recessed section, a lid part that is formed from a soft member through which an injection needle can be inserted and that closes the opening of the recessed section, a power reception coil that receives power sent from a power transmission coil, and a circuit board that is disposed in the main body at a position on the outer circumference side of the recessed section and has one surface on which a light-emitting unit for emitting light by means of the power received by the power reception coil is provided, wherein the circuit board extends along the outer circumference side of the recessed section in such an attitude that the surface on which the light-emitting unit is provided is oriented outward in the radial direction of the main body.

In the medical instrument according to the present invention, an emission direction in which light is emitted by the light-emitting unit is oriented toward an area above the main body with the circuit board being disposed on the outer circumference side of the recessed section.

In the medical instrument according to the present invention, the power reception coil is disposed between the lid part and the circuit board.

In the medical instrument according to the present invention, the power reception coil is disposed on the outer circumference side of the circuit board.

A medical device according to the present invention includes an outside-of-living-body unit that has a power transmission coil for sending power and a medical instrument that has a power reception coil for receiving the power sent from the outside-of-living-body unit and that is to be implanted in a living body, wherein the medical instrument includes a main body that has a recessed section extending in the thickness direction and having an upper surface with an opening and that is capable of storing a liquid in the recessed section, a lid part that is formed from a soft member through which an injection needle can be inserted and that closes the opening of the recessed section, and a circuit board that is disposed in the main body at a position on the outer circumference side of the recessed section and has one surface on which a light-emitting unit for emitting light by means of the power received by the power reception coil is provided, and the circuit board extends along the outer circumference side of the recessed section in such an attitude that the surface on which the light-emitting unit is provided is oriented outward in the radial direction of the main body.

### Effects of the Invention

In the present invention, the width direction of the circuit board is oriented in the thickness direction of the main body, and the outer diameter size of the circuit board in the radial direction of the main body can be small with the circuit board being attached to the main body, so that an installation surface for the light-emitting unit can be ensured, while the main body can have a reduced radial size.

### BRIEF DESCRIPTION OF THE DRAWINGS

FIG. 1 is a cross-sectional side view of a medical device according to one embodiment of the present invention;

FIG. 2 is a perspective view of a medical instrument according to one embodiment of the present invention;

FIG. 3 is an exploded perspective view of a medical instrument according to one embodiment of the present invention;

FIG. 4 is a schematic view of a circuit board according to one embodiment of the present invention;

FIG. 5 is a cross-sectional side view of a medical instrument according to another embodiment of the present invention;

FIG. 6 is a circuit diagram illustrating an example of the power reception circuit of the medical instrument of the present invention;

FIG. 7 is a circuit diagram illustrating another example of the power reception circuit of the medical instrument of the present invention;

FIG. 8 is a circuit diagram illustrating another example of the power reception circuit of the medical instrument of the present invention;

FIG. 9 is a circuit diagram illustrating another example of the power reception circuit of the medical instrument of the present invention;

FIG. 10 is a cross-sectional side view of a principal portion and illustrates the structure of connection between the power reception coil and the circuit board of the present invention;

FIG. 11 is a side view of a principal portion and illustrates the structure of connection between the power reception coil and the circuit board of the present invention;

FIG. 12 is a cross-sectional plan view illustrating the structure of connection between the power reception coil and the circuit board of the present invention; and

FIG. 13 is a side view of a principal portion and illustrates the structure of connection between the power reception coil and the circuit board of the present invention.

### PREFERRED MODE FOR CARRYING OUT THE INVENTION

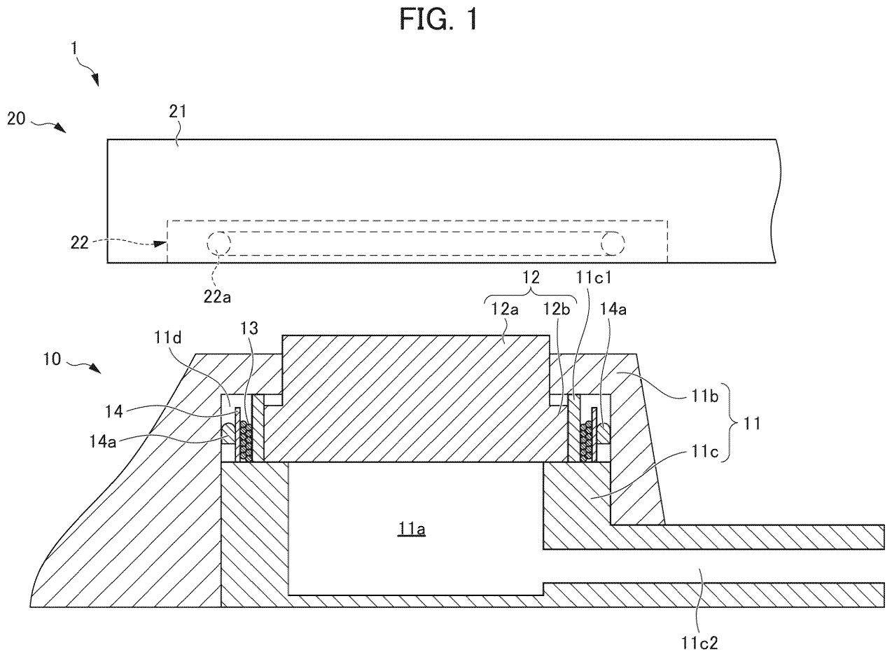

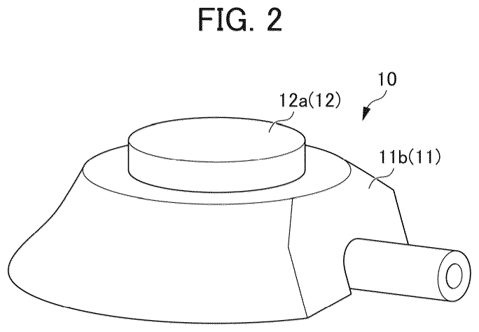

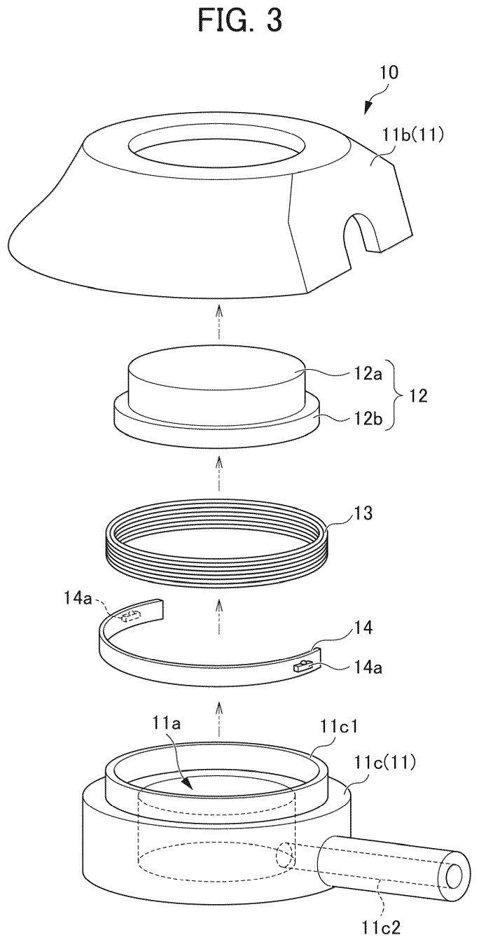

FIGS. 1 to 3 depict one embodiment of the present invention. FIG. 1 is a cross-sectional side view of the medical device. FIG. 2 is a perspective view of the medical instrument. FIG. 3 is an exploded perspective view of the medical instrument.

As depicted in FIG. 1, a medical device 1 of the present embodiment includes a medical instrument 10 that is used in a state of being implanted in a living body such as a human or an animal, and an outside-of-living-body unit 20 for supplying power to the medical instrument 10. The medical device 1 supplies power from the outside-of-living-body unit 20 to the medical instrument 10 in a noncontact manner.

The medical instrument 10 is a subcutaneous port (CV port), i.e., a type of central vein catheter, for injecting a medical solution into a central vein located in the vicinity of the heart of a living body. As depicted in FIGS. 1 to 3, the medical instrument 10 includes: a main body 11 that has a recessed section 11a in which a liquid such as a medical solution can be stored; a lid part 12 that closes the opening of the recessed section 11a of the main body 11; a power reception coil 13 that receives power sent from the outside-of-living-body unit 20; a circuit board 14 that is disposed in the main body 11 at a position on the outer circumference side of the recessed section 11a and has one surface on which a plurality of light-emitting units 14a for emitting light by means of the power received by the power reception coil 13 are provided; and a catheter (not illustrated) that has one end in communication with the recessed section 11a of the main body 11, and another end side to be inserted into a central vein of the living body.

For example, the main body 11 is formed from a resin material such as polyphenylene sulfide (PPS), polyethersulfone (PES), or polyetheretherketone (PEEK) and is formed in a substantially truncated cone shape. The main body 11 has formed therein the recessed section 11a, which has an upper surface with an opening and extends straight toward the bottom surface side. A liquid is stored in the recessed section 11a. The main body 11 is formed from an upper side member 11b and a lower side member 11c. The power reception coil 13 and the circuit board 14 are accommodated in a space 11d formed between the upper side member 11b and the lower side member 11c. The space 11d extends in such a manner as to assume a ring shape surrounding the outer circumference side of the recessed section 11a. The lower side member 11c has a liquid circulation passage 11c1 that is provided so as to extend from the bottom side toward the outer circumference side of the recessed section 11a and through which the liquid stored in the recessed section 11a circulates. One end of the catheter is connected to the liquid circulation passage 11c1. The recessed section 11a may also be referred to as, for example, a medical solution tank.

The lid part 12 is formed from a soft member, e.g., silicone rubber, through which an injection needle can be repeatedly inserted. The lid part 12 has a column-shaped lid body 12a, and a flange section 12b provided circumferentially entirely on the outer circumference portion of the lid body 12a and extending outward. The lid body 12a is a portion through which an injection needle is repeatedly inserted from the outside of the living body. The flange section 12b is a portion situated in the space 11d of the main body 11.

For example, by repeatedly winding a conducting wire of copper, the power reception coil 13 is formed in a ring shape having an inner diameter size that is larger than the outer diameter size of the flange section 12b of the lid part 12. For example, the power reception coil 13 is formed in a ring shape having a thickness of approximately 0.4 mm and a length of approximately 1.2 mm in the axial direction by winding, in the radial direction, a conducting wire having an outer diameter size of 0.2 mm in such a manner as to form two loops and by winding the same in the axial direction in such a manner as to form six loops. The power reception coil 13 is disposed between the lid part 12 and the circuit board 14.

The circuit board 14 is a so-called flexible board formed by printing a wire of, for example, copper foil on an insulator that is a resin material shaped like a film. The circuit board 14 is formed in the shape of a flexible strip. For example, the thickness of the film-shaped insulator of the circuit board 14 is approximately 0.05 mm. The thickness of the smallest portion of the circuit board 14 is, for example, 0.1 mm. One surface of the circuit board 14 has disposed thereon a plurality of light-emitting units 14a arranged at intervals, and a resonance circuit (not illustrated) to which the plurality of light-emitting units 14a are connected. The circuit board 14 has such a width-direction size that the light-emitting units 14a can be installed thereon. The power reception coil 13 is connected to the circuit board 14. Power received by the power reception coil 13 is supplied to each of the plurality of light-emitting units 14a. Furthermore, the circuit board 14 extends along the outer circumference side of the recessed section 11a in such an attitude that the surface on which the plurality of light-emitting unit 14a are provided is oriented outward in the radial direction of the main body 11, with the circuit board 14 being attached to the main body 11. As depicted in FIG. 3, the circuit board 14 assumes an arc shape surrounding a portion of the recessed section 11a on the outer circumference side, with the circuit board 14 being attached to the main body 11.

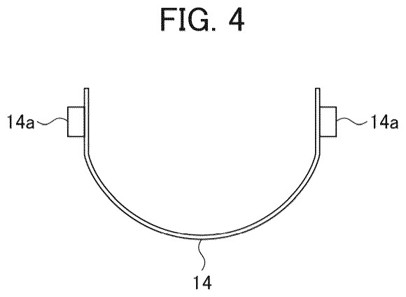

In this regard, forming the circuit board 14 formed from a flexible board into an arc shape to surround the recessed section 11a will result in loss of the flexibility of the portions of the circuit board 14 on which rigid electronic components such as the light-emitting units 14a are installed. Thus, the circuit board 14 formed from a flexible board is preferably formed such that, as depicted in FIG. 4: the portions on which the rigid electronic components are installed extend straight; and the portions other than the portions on which the rigid electronic components are installed are formed in an arc shape.

For example, the plurality of light-emitting units 14a are each a light emitting diode (LED), which emits light by having a voltage applied thereto. The plurality of light-emitting units 14a are each attached to the circuit board 14 such that an emission direction in which light is emitted is oriented toward an area above the main body 11 with the circuit board 14 being disposed on the outer circumference side of the recessed section 11a.

As depicted in FIG. 1, the outside-of-living-body unit 20 includes a housing 21 having such a size that a person can grip the same by hand, and a power transmission unit 22 provided in the housing 21. For example, the power transmission unit 22 has a power transmission coil 22a formed in a ring shape by repeatedly winding a conducting wire of copper, and a resonance circuit (not illustrated) to which the power transmission coil 22a is connected. Bringing the outside-of-living-body unit 20 close to the medical instrument 10 causes resonance to occur between the power transmission coil 22a of the power transmission unit 22 and the power reception coil 13 of the medical instrument 10, thereby transmitting power from the power transmission coil 22a to the power reception coil 13.

In the medical device 1 configured as described above, the medical instrument 10 is used in a state of being implanted in a living body. A liquid such as a medical solution to be administered into the living body is injected into the recessed section 11a of the main body 11 via an injection needle such as a Huber-pointed needle inserted through the lid part 12 from the outside of the living body. The liquid injected into the recessed section 11a of the main body 11 will pass through the liquid circulation passage 11c1, circulate through a catheter, and be administered into a central vein of the living body.

For the medical instrument 10 implanted in the living body, the position of the lid part 12 in the living body needs to be identified when the injection needle is inserted through the lid part 12. Thus, when inserting the injection needle through the lid part 12 of the medical instrument 10 implanted in the living body, the outside-of-living-body unit 20 is brought, from the outside of the living body, close to the portion in which the medical instrument 10 is implanted, and electricity is caused to flow through the power transmission coil 22a. As a result, the power transmission coil 22a of the outside-of-living-body unit 20 is brought close to the power reception coil 13 of the medical instrument 10 such that power is transmitted from the power transmission coil 22a to the power reception coil 13, and the plurality of light-emitting units 14a emit light by means of the power received by the power reception coil 13. The plurality of light-emitting units 14a each emit light toward an area above the main body 11 such that light is emitted upward from the outer circumference side of the lid part 12, and thus the light arrives at the outer circumference portion of the lid part 12 at the surface of the living body, thereby allowing the position of the lid part 12 to be identified. Under this condition, the injection needle can be reliably inserted through the lid part 12 by inserting the injection needle with a portion surrounded by the plurality of light-emitting units 14a as the target. Meanwhile, the power reception coil 13 and the circuit board 14 are situated on the lid part 12 side of the plurality of light-emitting units 14a. Accordingly, the light emitted from the light-emitting units 14a is blocked by the power reception coil 13 and the circuit board 14 and thus is not leaked to the lid part 12 side, and the outline of the lid part 12 can be clearly viewed from outside the living body.

As described above, according to the medical instrument of the present embodiment, a medical instrument 10 to be implanted in a living body includes a main body 11 that has a recessed section 11a extending in the thickness direction and having an upper surface with an opening and that is capable of storing a liquid in the recessed section 11a, a lid part 12 that is formed from a soft member through which an injection needle can be inserted and that closes the opening of the recessed section 11a, a power reception coil 13 that receives power sent from a power transmission coil 22a, and a circuit board 14 that is disposed in the main body 11 at a position on the outer circumference side of the recessed section 11a and has one surface on which a light-emitting unit 14a for emitting light by means of the power received by the power reception coil 13 is provided, wherein the circuit board 14 extends along the outer circumference side of the recessed section 11a in such an attitude that the surface on which the light-emitting unit 14a is provided is oriented outward in the radial direction of the main body 11.

The medical device of the present embodiment is a medical device 1 including an outside-of-living-body unit 20 that has a power transmission coil 22a for sending power and a medical instrument 10 that has a power reception coil 13 for receiving the power sent from the outside-of-living-body unit 20 and that is to be implanted in a living body, wherein the medical instrument 10 includes a main body 11 that has a recessed section 11a extending in the thickness direction and having an upper surface with an opening and that is capable of storing a liquid in the recessed section 11a, a lid part 12 that is formed from a soft member through which an injection needle can be inserted and that closes the opening of the recessed section 11a, and a circuit board 14 that is disposed in the main body 11 at a position on the outer circumference side of the recessed section 11a and has one surface on which a light-emitting unit 14a for emitting light by means of the power received by the power reception coil 13 is provided, and the circuit board 14 extends along the outer circumference side of the recessed section 11a in such an attitude that the surface on which the light-emitting unit 14a is provided is oriented outward in the radial direction of the main body 11.

Accordingly, the width direction of the circuit board 14 is oriented in the thickness direction of the main body 11, and the outer diameter size of the circuit board 14 in the radial direction of the main body 11 can be small with the circuit board 14 being attached to the main body 11, so that an installation surface for the light-emitting unit 14a can be ensured, while the main body 11 can have a reduced radial size.

With respect to the light-emitting units 14a, the emission direction in which light is emitted is preferably oriented toward an area above the main body 11 with the circuit board 14 being disposed on the outer circumference side of the recessed section 11a.

As a result, light emitted from the light-emitting units 14a with the medical instrument 10 being implanted in the living body can directly arrive at the surface of the living body, so that the lid part 12 can be reliably viewed from outside the living body.

The power reception coil 13 is preferably disposed between the lid part 12 and the circuit board 14.

In this way, the power reception coil 13 and the circuit board 14 can be formed as a single component, and the man-hours in the assembling work can be reduced.

FIG. 4 illustrates another embodiment of the present invention and is a cross-sectional side view of a medical instrument. Component parts identical to those in the embodiment noted above are given like reference marks.

With respect to a medical instrument 10 of the present embodiment, a power reception coil 13 is disposed on the outer circumference side of a circuit board 14 with the circuit board 14 being attached to a main body 11, and the medical instrument 10 is disposed below a plurality of light-emitting units 14a disposed on the outer peripheral surface of the circuit board 14. The power reception coil 13 in the present embodiment is formed in a ring shape having a larger inner diameter size than the circuit board 14 in a state of being attached to the main body 11. For example, the power reception coil 13 is formed in a ring shape having a thickness of approximately 0.8 mm and a length of approximately 0.6 mm in the axial direction by winding, in the radial direction, a conducting wire having an outer diameter size of 0.2 mm in such a manner as to form four loops and by winding the same in the axial direction in such a manner as to form three loops.

In the medical instrument 10 configured as described above, as in the embodiment noted above, light emitted from each of the plurality of light-emitting units 14a travels toward an area above the main body 11 in the thickness direction and arrives at the outer circumference portion of the lid part 12 at the surface of a living body, thereby allowing the position of the lid part 12 to be identified.

According to the medical instrument and the medical device of the present embodiment, accordingly, as in the embodiment noted above, the width direction of the circuit board 14 is oriented in the thickness direction of the main body 11, and the outer diameter size of the circuit board 14 in the radial direction of the main body 11 can be small with the circuit board 14 being attached to the main body 11, so that an installation surface for the light-emitting units 14a can be ensured, while the main body 11 can have a reduced radial size.

Meanwhile, the power reception coil 13 is preferably disposed on the outer circumference side of the circuit board 14.

In this way, the power reception coil 13 and the circuit board 14 can be formed as a single component, and the man-hours in the assembling work can be reduced.

In the embodiment noted above, the orientation of the circuit board 14 in the width direction is parallel to the thickness direction of the main body 11. However, the present invention is not limited to this. The orientation of the circuit board in the width direction does not need to be precisely parallel to the thickness direction of the main body, but may be inclined with respect to the thickness direction of the main body.

In the embodiment noted above, the circuit board 14 has such a length as to assume an arc shape surrounding a portion of the recessed section 11a on the outer circumference side with the circuit board 14 being attached to the main body 11. However, the present invention is not limited to this, and the circuit board 14 may have such a length as to assume a ring shape circumferentially entirely surrounding the outer circumference side of the recessed section 11a.

In the embodiment noted above, an LED is indicated as the light-emitting unit 14a. However, the light-emitting unit 14a is not limited to an LED, as long as the same emits light by means of power received by the power reception coil 13.

In the embodiment noted above, a plurality of light-emitting units 14a are arranged at intervals on the outer circumference side of the lid part 12 so that the position of the lid part 12 can be identified. However, the present invention is not limited to this. For example, one light-emitting unit that emits light assuming a ring shape to surround the outer circumference side of the lid part may be used, as long as the same allows the position of the lid part to be identified.

In the embodiment noted above, power is transmitted from the outside-of-living-body unit 20 to the medical instrument 10 by means of so-called magnetic resonance produced by a resonance circuit formed on the circuit board 14. However, power may be transmitted by electromagnetic induction.

In this regard, a power reception circuit formed in the medical instrument receives AC power irrespective of whether power is transmitted by electromagnetic induction or by magnetic resonance. In order to allow LEDs to emit light efficiently by means of power received from the power transmission coil, a full-wave rectifying circuit may be formed in the power reception circuit so as to convert the power into a direct current by full-wave rectification, or a plurality of LEDs having a rectification effect may be connected in parallel to the power reception circuit such that some of the plurality of LEDs are connected in the forward direction of the power reception circuit while the other LEDs are connected in the backward direction.

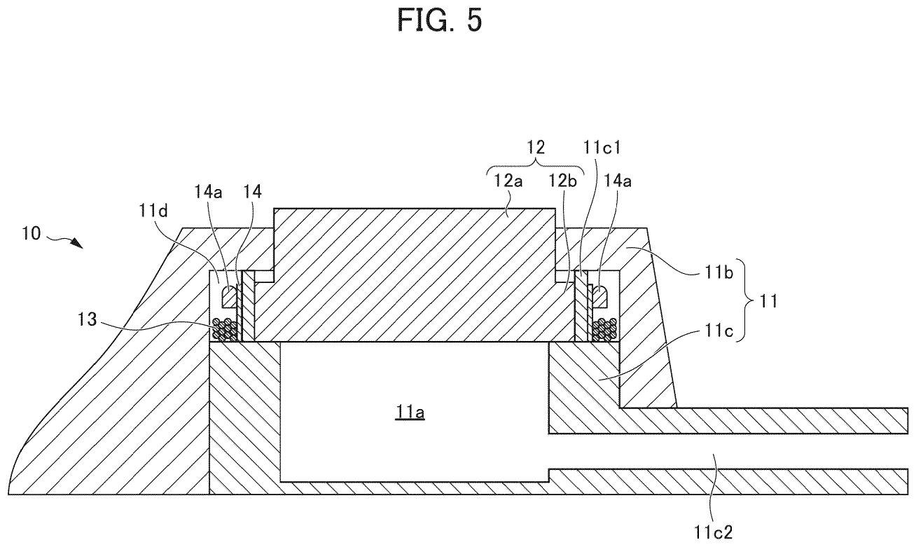

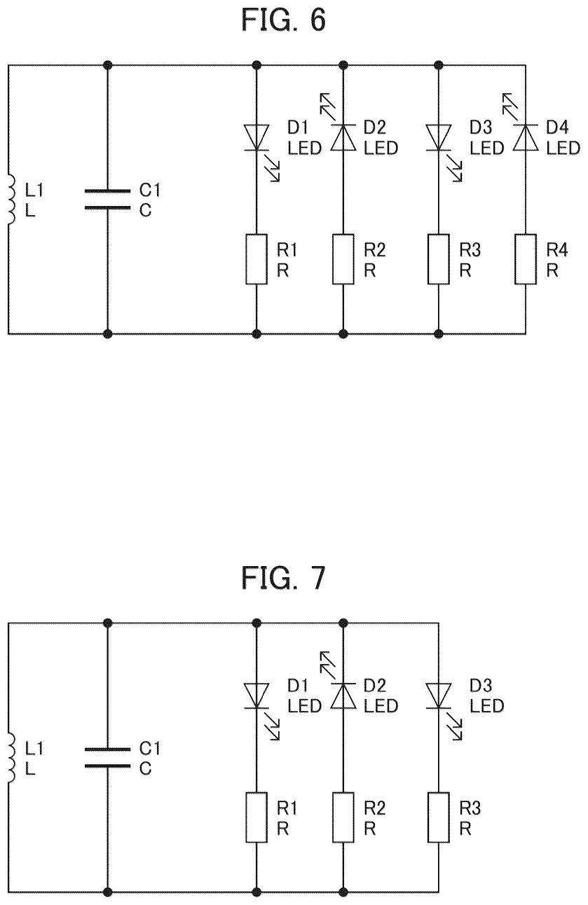

When an even number of LEDs are connected to the power reception circuit, the LEDs can be equal in brightness by making it so that, as depicted in FIG. 6, the number of LEDs connected in the forward direction of the power reception circuit is equal to the number of LEDs connected in the backward direction. In this way, the power reception circuit can be formed from fewer components, thereby reducing the manufacturing cost. Meanwhile, the brightness of some of the LEDs can be changed by adjusting the magnitude of a resistor to be installed in the power reception circuit.

In a case where an odd number of LEDs are connected to the power reception circuit, when the plurality of LEDs do not need to be the same in brightness, the number of LEDs connected in the forward direction of the power reception circuit may be different from that in the backward direction thereof, as depicted in FIG. 7.

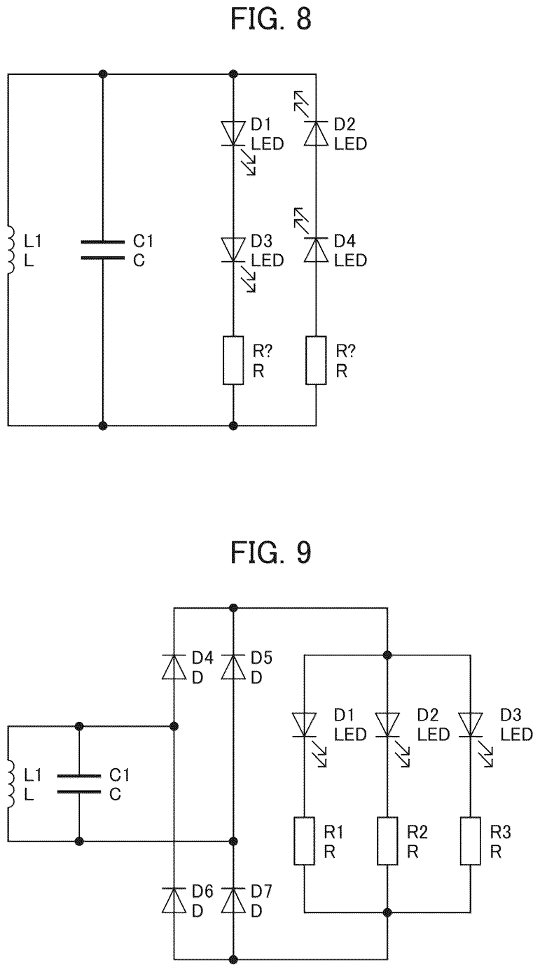

The plurality of LEDs may be connected in parallel to the power reception circuit as depicted in FIGS. 6 and 7, or may be connected in series thereto as depicted in FIG. 8. However, the plurality of LEDs are preferably connected in parallel to the power reception circuit, because a high voltage would be required to emit light if the same are connected in series to the power reception circuit.

In a case where an odd number of LEDs are connected to the power reception circuit, in order to make the plurality of LEDs equal in brightness, a full-wave rectifying circuit may be formed by arranging, as depicted in FIG. 9, four diodes in a bridge formation in the power reception circuit. In this situation, diodes having a low ON voltage, such as Schottky barrier diodes, are preferably used.

The power reception coil 13 and the circuit board 14 may be surface-treated with a coating material. It is preferable that the coating material used in this case is transparent with respect to visible light and has high biocompatibility.

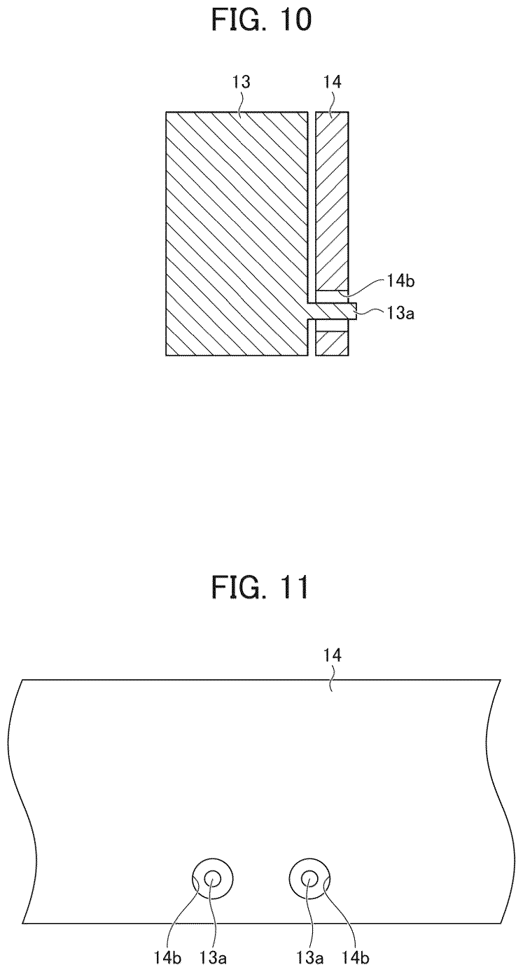

In order to connect the power reception coil 13 to the circuit board 14, an end portion of the conducting wire forming the power reception coil 13 may be connected directly to a printed wire of the circuit board 14, or, as depicted in FIGS. 10 and 11, a through-hole 14b may be formed in the circuit board 14, and an end portion 13a of the conducting wire of the power reception coil 13 may be connected to a printed wire of the circuit board 14 via the through-hole 14b. In this case, the conducting wire of the power reception coil 13 does not bulge from the surface of the circuit board 14, so that spaces to be occupied by the power reception coil 13 and the circuit board 14 can be reduced, and the medical instrument 10 can have a reduced size. Alternatively, in order to connect the power reception coil 13 to the circuit board 14, a notch section notching a width-direction end portion of the circuit board 14 into, for example, a semicircular shape may be formed without forming the through-hole 14b in the circuit board 14, and the power reception coil 13 may be connected to a printed wire of the circuit board 14 via the notch section. When connecting the power reception coil 13 to the circuit board 14, the position at which the conducting wire of the power reception coil 13 is connected to the strip-shaped circuit board 14 may be an end portion or an intermediate portion of the circuit board 14 in the longitudinal direction.

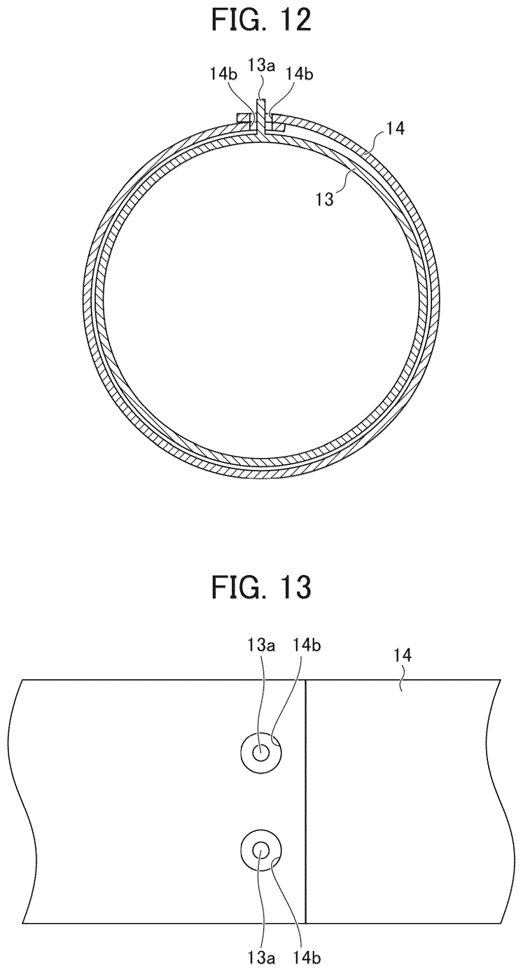

The circuit board 14 is not limited to one that partially surrounds the outer circumference portion of the power reception coil 13, but may be one circumferentially entirely surrounding the outer circumference portion of the power reception coil 13, as depicted in FIG. 12. In this case, as indicated in FIGS. 12 and 13, through-holes 14b are formed on the two end sides of the circuit board 14 in the longitudinal direction, and, with the through-holes 14b on the two sides in the longitudinal direction overlapping each other, the conducting wire of the power reception coil 13 is connected to a printed wire of the circuit board 14 via the through-holes 14b. As a result, the ring shape of the circuit board 14 can be maintained, thereby facilitating the assembling work for the medical instrument 10.

### EXPLANATION OF REFERENCE NUMERALS

1: Medical device
10: Medical instrument
11: Main body
11a: Recessed section
12: Lid part
13: Power reception coil
14: Circuit board
14a: Light-emitting unit
20: Outside-of-living-body unit
22a: Power transmission coil

1. A medical instrument to be implanted in a living body, the medical instrument comprising:
a main body that has a recessed section extending in a thickness direction and having an upper surface with an opening and that is capable of storing a liquid in the recessed section;
a lid part that is formed from a soft member through which an injection needle can be inserted and that closes the opening of the recessed section;
a power reception coil that receives power sent from a power transmission coil; and
a circuit board that is disposed in the main body at a position on an outer circumference side of the recessed section and has one surface on which a light-emitting unit for emitting light by means of the power received by the power reception coil is provided, wherein
the circuit board extends along the outer circumference side of the recessed section in such an attitude that the surface on which the light-emitting unit is provided is oriented outward in a radial direction of the main body.

2. The medical instrument according to claim 1, wherein
an emission direction in which light is emitted by the light-emitting unit is oriented toward an area above the main body with the circuit board being disposed on the outer circumference side of the recessed section.

3. The medical instrument according to claim 1, wherein
the power reception coil is disposed between the lid part and the circuit board.

4. The medical instrument according to claim 1, wherein
the power reception coil is disposed on an outer circumference side of the circuit board.

5. A medical device comprising an outside-of-living-body unit that has a power transmission coil for sending power and a medical instrument that has a power reception coil for receiving the power sent from the outside-of-living-body unit and that is to be implanted in a living body, wherein
the medical instrument includes
a main body that has a recessed section extending in a thickness direction and having an upper surface with an opening and that is capable of storing a liquid in the recessed section,
a lid part that is formed from a soft member through which an injection needle can be inserted and that closes the opening of the recessed section, and
a circuit board that is disposed in the main body at a position on an outer circumference side of the recessed section and has one surface on which a light-emitting unit for emitting light by means of the power received by the power reception coil is provided, wherein
the circuit board extends along the outer circumference side of the recessed section in such an attitude that the surface on which the light-emitting unit is provided is oriented outward in a radial direction of the main body.
